(19) United States
(12) Patent Application Publication
(10) Pub. No.: US 2008/0181920 A1
(43) Pub. Date: Jul. 31, 2008

(54) COSMETIC PREPARATION AGAINST SKIN PIGMENTATION

(75) Inventors: Anette Buerger, Hamburg (DE); Silke Heinecke, Hamburg (DE); Alexander Filbry, Hamburg (DE); Rainer Wolber, Hamburg (DE); Ludger Kolbe, Dohren (DE)

Correspondence Address: GREENBLUM & BERNSTEIN, P.L.C., 1950 ROLAND CLARKE PLACE, RESTON, VA, 20191, US

(73) Assignee: BEIERSDORF AG, Hamburg (DE)

(21) Appl. No.: 11/680,297

(22) Filed: Feb. 28, 2007

(30) Foreign Application Priority Data
Jan. 25, 2007 (DE) 10 2007 005 093.5

(51) Int. Cl. A61K 8/27; A61K 8/02; A61K 8/29; A61P 17/18; A61P 29/00; A61Q 17/04

(52) U.S. Cl. 424/401; 424/59; 424/63

(57) ABSTRACT

A cosmetic preparation comprising one or more compounds selected from salicylic acid and salts thereof and glycolic acid and salts thereof and one or more pigments selected from titanium dioxide pigments and zinc oxide pigments. This Abstract is not intended to define the invention disclosed in the specification, nor intended to limit the scope of the invention in any way.

### CROSS-REFERENCE TO RELATED APPLICATIONS

The present application claims priority under 35 U.S.C. § 119 of German Patent Application No. 10 2007 005 093.5, filed Jan. 25, 2007, the entire disclosure whereof is expressly incorporated by reference herein.

### BACKGROUND OF THE INVENTION

1. Field of the Invention

The present invention relates to a cosmetic preparation which comprises a combination of salicylic acid and/or glycolic acid and/or salts thereof and one or more pigments selected from titanium dioxide and zinc oxide pigments.

2. Discussion of Background Information

Human skin can take on different shades through pigmentation. Pigmentation of the skin is due to melanocytes, which are found in the bottom layer of the epidermis, the basal stratum, next to the basal cells, which—depending on skin type—are present as pigment-forming cells either individually or in relatively large numbers. Melanocytes contain melanosomes as characteristic cell organelles in which melanin is produced. More melanin is produced, inter alia, through stimulation by UV radiation. The melanin is transported via the living layers of the epidermis (keratinocytes) ultimately into the horny layer of the epidermis (corneocytes) and causes more or less pronounced brownish to brownish black skin coloring. Melanin is formed as the final stage in an oxidation process in which tyrosine, with the aid of the enzyme tyrosinase, is converted via several intermediate steps into the brown to brownish-black eumelanins (DHICA and DHI melanin) or with the involvement of sulfur-containing compounds to the reddish pheomelanin. DHICA and DHI melanin are produced by the common intermediate steps dopaquinone and dopachrome. The latter, in part with the involvement of further enzymes, is converted either into indole-5,6-quinone carboxylic acid or into indole-5,6-quinone, from which the two referenced eumelanins are produced. The production of pheomelanin takes place, inter alia, through the intermediate products dopaquinone and cysteinyl dopa.

Similar to the pigmentation of the skin, melanin-producing melanocytes are also responsible for hair color (pigmentation of the hair). The quantity and composition of the melanin in the hair determines the natural color of the hair, which is genetically determined.

Problems with hyperpigmentation (also referred to as over-pigmentation) of the skin have various causes and/or are side effects of many biological processes, e.g., UV radiation (e.g. freckles, ephelides), genetic disposition, defective pigmentation of the skin during scarring and/or healing of wounds, or skin aging (e.g. lentigines seniles).

Active substances and preparations are known that counteract the skin pigmentation. In practical use these are essentially preparations based on hydroquinone, which on the one hand do not show an effect until after several weeks of use, and on the other hand their excessively long use gives cause for concern for toxicological reasons.

The inhibition of the tyrosinase with substances such as kojic acid, ascorbic acid, and azelaic acid and their derivatives is also common. Furthermore, dicarboxylic acids are used to treat hyperpigmentations.

Conventional cosmetics or dermatics that contain active substances for lightening the skin (i.e., for treating hyperpigmentation) have a number of disadvantages:
The balance of effectiveness and tolerance is often inadequate. Either the preparations are insufficiently effective or insufficiently tolerated by the skin.
The production of preparations of this type is often complex and expensive.
Many of the formulations are even potentially toxic/carcinogenic and thus not suitable for long-term use.

WO 99/47118, US 2005/0019356, US 2005/0069566 disclose examples of corresponding cosmetic compositions.

It would be desirable to have available a combination of active substances that, incorporated into cosmetic preparations, significantly reduces or eliminates the disadvantages of the prior art.

### SUMMARY OF THE INVENTION

The present invention provides a cosmetic preparation comprising

(a) one or more compounds selected from salicylic acid and salts thereof (e.g., the alkali and alkaline earth metal salts such as, e.g., the Na, K, Mg, Ca salts) and glycolic acid and salts thereof (e.g., the alkali and alkaline earth metal salts);
(b) one or more pigments selected from titanium dioxide pigments and zinc oxide pigments.

In one aspect, the preparation may comprise component (a) in a total concentration of from about 0.05% to about 10% by weight, e.g., from about 0.1% to about 4% by weight, based on the total weight of the preparation.

In another aspect, the preparation may comprise salicylic acid and/or a salt thereof.

In another aspect, the preparation may comprise component (b) in a total concentration of from about 0.05% to about 10% by weight, based on the total weight of the preparation.

In yet another aspect, the preparation may comprise one or more titanium dioxide pigments in a total concentration of from about 0.1% to about 10% by weight, e.g., from about 0.5% to about 6% by weight, based on the total weight of the preparation.

In another aspect, component (b) may comprise one or more titanium dioxide pigments having a rutile structure and/or an average particle size of from about 10 to about 100 nm and/or a specific surface area of from about 30 to about 110 m²/g.

In another aspect, component (b) may comprise one or more titanium dioxide pigments which have a rutile structure and a specific surface area of from about 70 to about 120 m²/g and are coated with alumina and/or simethicone.

In another aspect, the preparation may further comprise one or more UV filter substances.

In another aspect, the preparation may further comprise one or more antioxidants in a total concentration of from about 0.01% to about 10% by weight, e.g., from about 0.01% to about 5% by weight, based on the total weight of the preparation. For example, the one or more antioxidants may comprise one or more of hydroquinone, a hydroquinone derivative (e.g., ubiquinone), tocopherol, a tocopheryl compound, ascorbic acid and an ascorbyl compound.

In another aspect, the preparation may further comprise one or more melanogenesis reducing substances in a total concentration of from about 0.01% to about 10% by weight, e.g., from about 0.1% to about 2% by weight, based on the total weight of the preparation. For example, the one or more melanogenesis reducing substances may comprise at least one substance selected from octadecenedioic acid, kojic acid, soy, α-lipoic acid, retinoids, azelaic acid and phenolic compounds.

In yet another aspect, the preparation may further comprise one or more anti-inflammatory substances in a concentration of from about 0.01% to about 5% by weight, e.g., from about 0.01% to about 3% by weight, based on the total weight of the preparation. For example, the one or more anti-inflammatory substances may comprise at least one extract of glycyrrhiza inflata, chamomilla recutita and/or arctiium lappa.

In a still further aspect, the preparation may comprise

(i) one or more titanium dioxide pigments in a concentration of from about 0.1% to about 10% by weight;
(ii) salicylic acid and/or a salt thereof in a concentration of from about 0.05% to about 10% by weight;
(iii) one or more antioxidants selected from ubiquinone, tocopherol and tocopherol derivatives in a concentration of from about 0.005% to about 7% by weight;
(iv) octadecenedioic acid in a concentration of from about 0.05% to about 5% by weight;
each based on the total weight of the preparation.

In another aspect, the preparation may comprise

(i) one or more titanium dioxide pigments having a rutile structure in a concentration of from about 0.1% to about 10% by weight;
(ii) salicylic acid and/or a salt thereof in a concentration of from about 0.1% to about 2% by weight;
(iii) ubiquinone in a concentration of from about 0.01% to about 0.5% by weight;
(iv) octadecenedioic acid in a concentration of from about 0.1% to about 2% by weight;
(v) glycyrrhiza inflata in a concentration of from about 0.01% to about 1% by weight;
each based on the total weight of the preparation.

In another aspect, the preparation may comprise one or more, e.g., two or more or three or more, of ethylhexyl methoxycinnamate, ethylhexyl salicylate, benzophenone-3, phenylbenzimidazole sulfonic acid and homosalate.

The present invention also provides a cosmetic preparation comprising

(a) from about 0.1% to about 4% by weight of one or more compounds selected from salicylic acid and salts thereof;
(b) from about 0.5% to about 6% by weight of one or more titanium dioxide pigments;
each based on the total weight of the preparation.

In one aspect of the preparation, component (b) may comprise one or more titanium dioxide pigments having a rutile structure and an average particle size of from about 10 to about 100 nm.

In another aspect, component (b) may comprise one or more titanium dioxide pigments having a specific surface area of from about 30 to about 110 m²/g.

In yet another aspect, component (b) may comprise one or more titanium dioxide pigments which have a rutile structure and a specific surface area of from about 70 to about 120 m²/g and are coated with alumina and simethicone.

In another aspect, the preparation may further comprise one or more antioxidants selected from ubiquinone, tocopherol and tocopherol derivatives in a total concentration of from about 0.01% to about 10% by weight and/or one or more melanogenesis reducing substances selected from octadecenedioic acid, kojic acid, soy, α-lipoic acid, retinoids, azelaic acid and phenolic compounds in a total concentration of from about 0.01% to about 10% by weight, each based on a total weight of the preparation.

The present invention also provides a cosmetic preparation comprising

(a) from about 0.1% to about 4% by weight of one or more compounds selected from salicylic acid and salts thereof;
(b) from about 0.5% to about 6% by weight of one or more titanium dioxide pigments having a rutile structure and a specific surface area of from about 70 to about 130 m²/g; and at least one of
(c) from about 0.01% to about 5% by weight of one or more antioxidants selected from ubiquinone, tocopherol and tocopherol derivatives;
(d) from about 0.1% to about 2% by weight of one or more substances selected from octadecenedioic acid, kojic acid, soy, α-lipoic acid, retinoids, azelaic acid and phenolic compounds; and
(e) from about 0.01% to about 3% by weight of one or more anti-inflammatory substances;
each based on the total weight of the preparation.

The present invention also provides a method of preventing or treating skin pigmentation. The method comprises applying to the skin a cosmetic preparation according to the present invention.

### DETAILED DESCRIPTION OF THE PRESENT INVENTION

The particulars shown herein are by way of example and for purposes of illustrative discussion of the embodiments of the present invention only and are presented in the cause of providing what is believed to be the most useful and readily understood description of the principles and conceptual aspects of the present invention. In this regard, no attempt is made to show structural details of the present invention in more detail than is necessary for the fundamental understanding of the present invention, the description making apparent to those skilled in the art how the several forms of the present invention may be embodied in practice.

It is preferred according to the present invention for component (a) to comprise salicylic acid.

It is preferred to use titanium dioxide pigments with an average particle size of from about 10 to about 100 nm. It is particularly preferred to use titanium dioxide pigments with an average particle size of from about 15 to about 50 nm. According to the invention, it is particularly preferred to use titanium dioxide particles with rutile structure.

If zinc oxide pigments are used, it is advantageous according to the present invention to use zinc oxide pigments with an average particle size of from about 10 to about 250 nm.

The pigments (titanium dioxide, zinc oxide) may be employed advantageously in the form of commercially available oleaginous or aqueous predispersions. Dispersants and/or solubilizers may advantageously be present in these predispersions.

The pigments (titanium dioxide and/or zinc oxide) for the purposes of the present invention, may advantageously be surface-treated (“coated”), the intention being, for example, to form or retain a hydrophilic, amphiphilic or hydrophobic character. This surface treatment can comprise providing the pigments with a thin hydrophilic and/or hydrophobic inorganic and/or organic layer by processes known per se. For the purposes of the present invention, the different surface coatings can also comprise water.

Inorganic surface coatings for the purposes of the present invention may comprise, for example, aluminum oxide (Al₂O₃), aluminum hydroxide Al(OH)₃, aluminum oxide hydrate (also: alumina, CAS No.: 1333-84-2), sodium hexametaphosphate (NaPO₃)₆, sodium metaphosphate (NaPO₃)ₙ, silicon dioxide (SiO₂) (also: silica, CAS No.: 7631-86-9), barium sulfate (BaSO₄) and/or iron oxide (Fe₂O₃). These inorganic surface coatings may be present on their own, in combination and/or in combination with organic coating materials.

Organic surface coatings for the purposes of the present invention may comprise, for example, one or more of vegetable or animal aluminum stearate, vegetable or animal stearic acid, lauric acid, dimethylpolysiloxane (also: dimethicone), methylpolysiloxane (methicone), simethicone (a mixture of dimethylpolysiloxane with an average chain length of from about 200 to about 350 dimethylsiloxane units and silica gel) and alginic acid. These organic surface coatings may be present on their own, in combination and/or in combination with inorganic coating materials.

The preferred titanium dioxide pigments for use in the present invention have a specific surface area of from about 30 to about 110 m²/g (measured by the BET method according to DIN ISO 9277:2003-05 (BET method) or DIN 66132: 1975-07).

Particularly preferred titanium dioxide pigments have dimensions of about 35 nm×29 nm or about 77 nm×22 nm.

According to the invention, it is most preferred to use titanium dioxide pigments that have a rutile structure and a specific surface area of from about 70 to about 120 m²/g and are surface-coated with alumina/simethicone.

It is advantageous for the preparation or use according to the present invention to further comprise one or more UV photoprotective filters selected, e.g., from phenylene-1,4-bis-(2-benzimidazyl)-3,3′-5,5′-tetrasulfonic acid salts; 2-phenylbenzimidazole-5-sulfonic acid salts; 1,4-di(2-oxo-10-sulfo-3-bomylidenemethyl)-benzene und salts thereof; 4-(2-oxo-3-bomylidenemethyl)benzenesulfonic acid salts; 2-methyl-5-(2-oxo-3-bomylidenemethyl)sulfonic acid salts; 2,2′-methylene-bis-(6-(2H-benzotriazole-2-yl)-4-(1,1,3,3-tetramethylbutyl)-phenol); 2-(2H-benzotriazole-2-yl)-4-methyl-6-[2-methyl-3-[1,3,3,3-tetramethyl-1-[(trimethylsilyl)oxy]disiloxanyl]propyl]-phenol; 3-(4-methylbenzylidene)campher; 3-benzylidene campher; ethylhexyl salicylate; terephthalidene dicamphorsulfonic acid; (2-ethylhexyl)4-(dimethylamino)benzoate; amyl 4-(dimethylamino)benzoate; di(2-ethylhexyl)4-methoxybenzalmalonate; (2-ethylhexyl)4-methoxycinnamate; isoamyl 4-methoxycinnamate; 2-hydroxy-4-methoxybenzophenone, 2-hydroxy-4-methoxy-4′-methylbenzophenone; 2,2′-dihydroxy-4-methoxybenzophenone; hexyl 2-(4′-diethylamino-2′-hydoxybenzoyl)benzoate, 4-(tert.-butyl)-4′-methoxydibenzoylmethane; momomenthylsalicylate; 2-ethylhexyl 2-hydroxybenzoate; 2-ethylhexyl-2-cyano-3,3-diphenylacrylate; dimethicodiethylbenzalmalonate; 3-(4-(2,2-bis-ethoxycarbonylvinyl)-phenoxy)propenyl)-methoxysiloxane/dimethylsiloxane copolymer; 2,4-bis-{[4-(2-ethylhexyloxy)-2-hydroxy]-phenyl}-6-(4-methoxyphenyl)-1,3,5-triazine (INCI: bis-ethylhexyloxyphenol methoxyphenyl triazine); dioctylbutylamidotriazone (INCI: diethylhexyl-butamidotriazone); 2,4-bis-[5-1 (dimethylpropyl)benzoxazole-2-yl-(4-phenyl)-imino]-6-(2-ethylhexyl)-imino-1,3,5-triazine (CAS Nr. 288254-16-0); tris(2-ethylhexyl)4,4′,4″-(1,3,5-triazine-2,4,6-triyltriimino)-tris-benzoate (also: 2,4,6-tris-[anilino-(p-carbo-2′-ethyl-1′-hexyloxy)]-1,3,5-triazine (INCI: ethylhexyl triazone); 2,4,6-tribiphenyl-4-yl-1,3,5-triazine; merocyanine; benzoylcinnamic acid nitriles, preferably in a concentration of from about 0.01% to 40% by weight, based on the total weight of the preparation.

It is particularly preferred according to the present invention if the preparation additionally comprises one or more UVB filters.

It is advantageous for the preparation or method according to the present invention if the preparation further comprises one or more antioxidants in a total concentration of from about 0.01% to about 10% by weight, preferably from about 0.01% to about 5% by weight, based on the total weight of the preparation.

Examples of preferred antioxidants include one or more of hydroquinone, hydroquinone derivatives (e.g., ubiquinone), tocopherol, tocopheryl compounds, ascorbic acid and ascorbyl compounds. Ubiquinone, tocopherol and tocopheryl acetate are particularly preferred.

Furthermore, it is advantageous for the preparation or method according to the present invention if the preparation further comprises one or more melanogenesis reducing substances in a total concentration of from, e.g., about 0.01% to 10% by weight, preferably from about 0.1% to about 2% by weight, based on the total weight of the preparation. Preferred examples of melanogenesis reducing substances include substances selected from octadecenedioic acid, kojic acid, soy, α-lipoic acid, retinoids, azelaic acid and phenolic compounds. Octadecenedioic acid is a particularly preferred melanogenesis reducing substance for the purposes of the present invention.

Furthermore, it is advantageous for the preparation or method according to the present invention if the preparation further comprises one or more anti-inflammatory active substances in a total concentration of from, e.g., about 0.01% to about 5% by weight, preferably from about 0.01% to about 3% by weight, based on the total weight of the preparation. Examples of preferred anti-inflammatory active substances include plant extracts of, e.g., glycyrrhiza inflata, chamomilla recutita or arctiium lappa which comprise anti-inflammatory compounds or the active substance extracts thereof or the group of NSARs (non-steroidal antirheumatics). Particularly preferred according to the present invention are glycyrrhiza inflata root extract and arctiium lappa extract.

It may be advantageous to incorporate one or more of the above components into the preparation of the present invention in encapsulated form. As encapsulation agents, e.g., wax or lipid particles (e.g., of wax/oil mixtures) or cyclodextrins may advantageously be used. The wax or lipid particles may advantageously have a particle size of from about 0.2 to about 50 μm, preferably from about 0.4 to about 30 μm and particularly preferably from about 0.5 to about 25 μm.

The preparation according to the present invention may advantageously be an aqueous solution, a gel or an oil-in-water emulsion (O/W emulsion). Silicone oil-in-water emulsions, W/O emulsions, W/O/W or O/W/O emulsions may also be used advantageously according to the invention. Such formulations may preferably be present in the form of a micro-emulsion (e.g., a PIT emulsion) or a solids emulsion (i.e. an emulsion which is stabilized by solids such as, e.g. a Pickering emulsion).

The preparation according to the present invention may advantageously be present in the form of an ointment, a cream, a lotion or an emulsion foam.

Particularly advantageously, the preparation according to the invention is present in the form of an emulsion.

It is particularly preferred for the preparation of the present invention to be present in the form of an O/W emulsion. In this case it is particularly preferred for the preparation to comprise one or more O/W emulsifiers selected from glyceryl stearate citrate, glyceryl stearate (self-emulsifying), stearic acid, stearate salts, polyglyceryl-3 methylglucose distearate, ceteareth-20, PEG-40 stearate, sodium cetearyl sulfate.

These O/W emulsifiers may advantageously be present in a concentration of from about 0.1% to about 10% by weight, preferably from about 0.2% to about 7% by weight, based on the total weigh of the preparation.

Moisturizers are substances or mixtures of substances which impart to cosmetic or dermatological preparations the property, following application or distribution on the surface of the skin, of reducing moisture release by the horny layer (also called trans-epidermal water loss (TEWL)) and/or of positively influencing hydration of the horny layer.

Examples of preferred moisturizers for the purposes of the present invention include glycerol, lactic acid and lactates, in particular sodium lactate, butylene glycol, propylene glycol, biosaccharide gum-1, glycine soya, ethylhexyloxyglycerol, pyrrolidonecarboxylic acid and urea. In addition, it is particularly advantageous to use one or more polymeric moisturizers from the group of water-soluble, water-swellable or water-gellable polysaccharides. Hyaluronic acid, chitosan and a fucose-rich polysaccharide which is filed in the Chemical Abstracts under the Registry Number 178463-23-5 and which is available, for example, under the name Fucogel® 1000 from SOLABIA S.A., are particularly advantageous moisturizers. Moisturizers can advantageously also be used as anti-wrinkle active ingredients for protection from changes in the skin, as arise, for example, during skin aging.

The preparation according to the present invention may advantageously comprise one or more moisturizers, e.g., in a total concentration of from about 0.1% to about 20% by weight, preferably from about 0.5% to about 10% by weight, based on the total weight of the preparation.

The cosmetic or dermatological preparations according to the invention can also advantageously, but not necessarily, comprise fillers which, for example, further improve the sensory and cosmetic properties of the formulations and, for example, bring about or enhance a velvety or silky feel on the skin. Examples of advantageous fillers for the purposes of the present invention include starch and starch derivatives (such as, for example, tapioca starch, distarch phosphate, aluminum or sodium starch octenylsuccinate and the like), pigments which have neither a primarily UV filter effect nor a coloring effect (such as, for example, boron nitride etc.) and Aerosils® (CAS No. 7631-86-9) and/or talc.

The aqueous phase of the preparations according to the present invention may also comprise one or more customary cosmetic auxiliaries, such as, e.g., alcohols, in particular those with a low carbon number, preferably ethanol and/or isopropanol, diols or polyols of low carbon number, and ethers thereof, preferably propylene glycol, 2-methyl-1,3-propanediol, glycerin, ethylene glycol, ethylene glycol monoethyl or monobutyl ether, propylene glycol monomethyl, monoethyl or monobutyl ether, diethylene glycol monomethyl or monoethyl ether and analogous products, polymers, foam stabilizers, electrolytes and in particular one or more thickeners, in particular, one or more thickeners which can advantageously be chosen from silicon dioxide, aluminum silicates, polysaccharides and derivatives thereof, e.g. hyaluronic acid, xanthan gum, hydroxypropylmethyl cellulose, particularly advantageously from polyacrylates, preferably a polyacrylate from the group of so-called carbopols, for example carbopols of the types 980, 981, 1382, 2984, 5984, either individually or in combination. Further thickeners that are advantageous according to the invention include Pemulen TR1, TR2 and Aristoflex AVC.

The oil phase of a preparation (e.g., emulsion) of the present invention may advantageously comprise one or more substances selected from polar oils, for example from the group of lecithins and of fatty acid triglycerides, namely the triglycerol esters of saturated and/or unsaturated, branched and/or unbranched alkanecarboxylic acids having a chain length of from about 8 to about 24, in particular about 12 to about 18, carbon atoms. The fatty acid triglycerides can, for example, be chosen advantageously from synthetic, semisynthetic and natural oils, such as, for example, cocoglyceride, olive oil, sunflower oil, soya oil, peanut oil, rapeseed oil, almond oil, palm oil, coconut oil, castor oil, wheatgerm oil, grapeseed oil, thistle oil, evening primrose oil, macadamia nut oil and the like.

Also advantageous according to the invention are, for example, natural waxes of animal and vegetable origin, such as, for example, beeswax and other insect waxes, and berry wax, shea butter and/or lanolin (wool wax).

For the purposes of the present invention, further advantageous polar oil components can also be chosen from the esters of saturated and/or unsaturated, branched and/or unbranched alkanecarboxylic acids of a chain length from about 3 to about 30 carbon atoms and saturated and/or unsaturated, branched and/or unbranched alcohols of a chain length from about 3 to about 30 carbon atoms, and from the esters of aromatic carboxylic acids and saturated and/or unsaturated, branched and/or unbranched alcohols of chain length from about 3 to about 30 carbon atoms. Non-limiting examples of such ester oils include phenylethyl benzoate, octyl palmitate, octyl cocoate, octyl isostearate, octyldodecyl myristate, octyldodecanol, cetearyl isononanoate, isopropyl myristate, isopropyl palmitate, isopropyl stearate, isopropyl oleate, n-butyl stearate, n-hexyl laurate, n-decyl oleate, isooctyl stearate, isononyl stearate, isononyl isononanoate, 2-ethylhexyl palmitate, 2-ethylhexyl laurate, 2-hexyldecyl stearate, 2-octyldodecyl palmitate, stearyl heptanoate, isopropyl lauroyl sarcosinate, oleyl oleate, oleyl erucate, erucyl oleate, erucyl erucate, tridecyl stearate, tridecyl trimellitate, and synthetic, semisynthetic and natural mixtures of such esters, such as, for example, jojoba oil.

In addition, the oil phase may advantageously be chosen from dialkyl ethers and dialkyl carbonates, advantageous examples thereof being dicaprylyl ether (Cetiol OE) and/or dicaprylyl carbonate, for example, that available under the trade name Cetiol CC from Cognis.

It is also preferred to choose the one or more oil components from isoeicosane, neopentyl glycol diheptanoate, propylene glycol dicaprylate/dicaprate, caprylic/capric/diglyceryl succinate, butylene glycol dicaprylate/dicaprate, C₁₂₋₁₃-alkyl lactate, di-C₁₂₋₁₃-alkyl tartrate, triisostearin, dipentaerythrityl hexacaprylate/hexacaprate, propylene glycol monoisostearate, tricaprylin, dimethyl isosorbide. It is particularly advantageous if the oil phase of the preparation of the present invention comprises C₁₂₋₁₅-alkyl benzoate, or is composed entirely thereof. Further examples of advantageous oil components are butyloctyl salicylate (for example that available under the trade name Hallbrite BHB from CP Hall), hexadecyl benzoate and butyloctyl benzoate and mixtures thereof (Hallstar AB).

Any mixtures of such oil and wax components can also be used advantageously for the purposes of the present invention.

In addition, the oil phase may advantageously also comprise nonpolar oils, for example those which are chosen from branched and unbranched hydrocarbons and hydrocarbon waxes, in particular mineral oil, vaseline (petrolatum), paraffin oil, squalane and squalene, polyolefins, hydrogenated polyisobutenes C₁₃₋₁₆ isoparaffin and isohexadecane. Among the polyolefins, polydecenes are the preferred substances.

The cosmetic preparations according to the present invention may comprise cosmetic auxiliaries such as those which are customarily used in such preparations, e.g. preservatives, preservative aids, complexing agents, bactericides, perfumes, substances for preventing or increasing foaming, dyes, pigments which have a coloring effect, thickeners, moisturizing and/or humectant substances, fillers which improve the feel on the skin, fats, oils, waxes and other customary constituents of a cosmetic or dermatological formulation, such as alcohols, polyols, polymers, foam stabilizers, electrolytes, organic solvents or silicone derivatives.

A preferred embodiment of a cosmetic preparation of the present invention comprises a combination of

(a) one or more titanium dioxide pigments in a concentration of from about 0.1% to about 10% by weight,
(b) salicylic acid and/or salts thereof in a concentration of from about 0.05% to about 10% by weight, preferably from about 0.1% to about 2% by weight,
(c) one or more antioxidants selected from ubiquinone, tocopherol and tocopherol derivatives in a concentration of from about 0.005% to about 7% by weight, preferably from about 0.01% to about 0.5% by weight,
(d) octadecenedioic acid in a concentration of from about 0.05% to about 5% by weight, preferably from about 0.1% to about 2% by weight,
each based on the total weight of the preparation.
Another preferred embodiment of a cosmetic preparation of the present invention comprises
(a) one or more titanium dioxide pigments in a concentration of from about 0.1% to about 10% by weight,
(b) salicylic acid and/or salts thereof in a concentration of from about 0.1% to about 2% by weight,
(c) ubiquinone in a concentration of from about 0.01% to about 0.5% by weight,
(d) octadecenedioic acid in a concentration of from about 0.1% to about 2% by weight,
(e) glycyrrhiza inflata extract in a concentration of from about 0.01% to about 1% by weight,
each based on the total weight of the preparation.

It is particularly preferred according to the present invention for the preferred coated titanium dioxide described above to be used in both of the above preferred embodiments.

### EXAMPLES

The following examples are intended to illustrate the present invention without restricting it. Unless stated otherwise, all quantities, proportions and percentages refer to the weight and the total amount or the total weight of the preparations.

### Examples 1-10

### O/W Creams

| | Example No. | | | | |
|---|---|---|---|---|---|
| | 1 | 2 | 3 | 4 | 5 |
| Glyceryl stearate citrate | 2 | | | 2 | |
| Glyceryl stearate, self-emulsifying | | 5 | 3 | | 2 |
| PEG-40 stearate | | | 1 | | 1 |
| Behenyl alcohol | | | | 2 | |
| Stearyl alcohol | 2 | 1 | | | |
| Cetearyl alcohol | | | | 4 | 2 |
| Cetyl alcohol | 1 | | 3 | | |
| Hydrogenated cocoglycerides | 2 | | | | |
| Shea buffer | | 2 | | | 2 |
| C_{12–15} Alkylbenzoate | | 3 | 2 | | 3 |
| Butylene glycol dicaprylate/dicaprate | 1 | | | 1 | |
| Caprylic/capric triglyceride | | 1 | 1 | 2 | 2 |
| Ethylhexyl coconut fatty acid ester | 3 | | | | 1 |
| Octyldodecanol | | | 1 | | |
| Mineral oil | | 1 | | | |
| Petrolatum | 2 | | 1 | | 2 |
| Octamethyltetrasiloxane (Cyclomethicone) | 4 | 1 | 4 | 3 | 5 |
| Dimethylpolysiloxane (Dimethicone) | | | 1 | 1 | |
| Dicaprylylether | 1 | 4 | 2 | | |
| ZnO | 4 | | | | |
| ZnO-Dimethicone | | 2 | 1 | | |
| TiO₂ | | | | 3 | |
| TiO₂ + alumina + silica | | | 2 | | 1 |
| Ethylhexyl methoxycinnamate | | | | | 2 |
| Ethylhexyl cyanodiphenylacrylate (Octocrylene) | | 3 | | | |
| Bis-ethylhexyloxyphenol methoxyphenyl triazine | | 0.5 | | | |
| Ethylhexyl salicylate | | | | | 1 |
| Ubiquinone (Q10) | 0.05 | | | | |
| Sodium ascorbyl phosphate | | | | | 0.04 |
| Tocopherol | | 1 | | 0.5 | |
| Retinol | | | 0.1 | | |
| Tocopheryl acetate | | | 1 | | |
| Citric acid, sodium salt | | 0.1 | | | |
| 8-Hexadecene-1,16-dicarboxylic acid | 0.1 | | 1 | | 0.2 |
| Kojic acid | 0.5 | | | | |
| Ascorbic acid | | 0.5 | | | |
| Hydroquinone | | | | 0.5 | |
| Glycine soya | 1 | | 2 | | |
| Glycyrrhiza inflata | 0.02 | | 0.025 | | 0.04 |
| Arctium lappa fruit extract | | 1 | | 2 | |
| Bisabolol | | | | | 0.5 |
| Iminodisuccinate, sodium salt | 0.2 | | 0.1 | | 0.1 |
| Phenoxyethanol | 0.3 | | 0.3 | 0.2 | 0.2 |
| Ethyl paraben | 0.6 | 0.3 | 0.2 | 0.3 | 0.3 |
| Hexamidine diisethionate | | 0.04 | | | |
| Diazolidinyl urea | 0.25 | | 0.1 | 0.2 | 0.1 |
| 1,3-Dimethylol-5,5-dimethylhydantoin (DMDM | | 0.2 | | | |
| hydantoin) | | | | | |
| Iodopropynylbutyl carbamate | | 0.1 | | | |
| Ethanol denatured | | 2 | | | |
| Xanthan gum | 0.1 | | | | |
| Polyacrylic acid (Carbomer) | 0.05 | | 0.1 | | 0.1 |
| Polyacrylamide | | 0.2 | | | |
| Glycerin | 10 | 6 | 6 | 7.5 | 8 |
| Butylene glycol | 2 | 1 | | | |
| Water-soluble and/or oil-soluble dyes | 0.05 | | | | |
| Fillers/additives (distarch phosphate, SiO₂, BHT, | 0.1 | 1 | 0.2 | 0.5 | 0.05 |
| talc, aluminum stearate) | | | | | |
| Perfume | q.s. | q.s. | q.s. | q.s. | q.s. |
| Water | ad | ad | ad | ad | ad 100 |
| | 100 | 100 | 100 | 100 | |

| | Example Number | | | | |
|---|---|---|---|---|---|
| | 6 | 7 | 8 | 9 | 10 |
| Glyceryl stearate, self-emulsifying | | | | | 1 |
| PEG-30 stearate | 1.5 | | | | |
| Glyceryl stearate | 4.5 | | | 3 | |
| Polyglyceryl-3-methylglucose distearate | | 3 | | | |
| PEG-40 castor oil | | 1 | | | 1 |
| Sodium cetearyl sulfate | | | 2 | | 0.5 |
| Polyethylene glycol(2)stearyl-ether (Steareth-2) | | | 1 | | |
| Cetearyl glucoside | | | | 2 | |
| Myristyl myristate | | | | 1 | |
| Cetyl alcohol | 1 | 1 | | | 2.5 |
| Stearyl alcohol | | | | 5 | |
| Cetearyl alcohol | | | 2 | | 1 |
| Alcohol denat. | 2 | | | | 2 |
| Hydrogenated cocoglycerides | 1.5 | | | 1 | 1 |
| Shea butter | | 2 | | | |
| C_{12–15} Alkylbenzoate | 3 | | 5 | 2 | |
| Butylene glycol dicaprylate/dicaprate | | | | 2 | 3 |
| Caprylic/capric triglyceride | | 1 | | 3 | |
| C_{18–36} Triglycerides | | | | 1 | 1 |
| Ethylhexyl coconut fatty acid ester | | | 2 | | |
| Octyldodecanol | | | 1 | | |
| Mineral oil | | | 1 | | |
| Petrolatum | | 1 | | | |
| Octamethyltetrasiloxane | 2 | 3 | 2 | | |
| (Cyclomethicone) | | | | | |
| Dimethylpolysiloxane | 3 | | | 1 | |
| (Dimethicone) | | | | | |
| Dicaprylylether | | | 2 | | |
| Dicarprylyl carbonate | | 2 | | 3 | |
| Polydecene | | | | 4 | |
| TiO₂ | | | 1 | | |
| TiO₂ + alumina + simethicone | 4 | | | | 4 |
| ZnO | | | 1 | 3 | |
| ZnO + dimethiconol | | 4 | | | |
| Ethylhexyl methoxycinnamate | 7.5 | 3 | | | 7.5 |
| Ethylhexyl salicylate | | | 2 | | 4.5 |
| Benzophenone-3 | 4 | | | | 4 |
| Phenylbenzimidazole sulfonic acid | 2 | | | 1 | |
| Homosalate | 4 | | | | 9.5 |
| Bis-ethylhexyloxyphenol methoxyphenyl | | | 1 | | |
| triazine | | | | | |
| Ubiquinone (Q10) | 0.03 | | | 0.3 | 0.03 |
| Biotin | | 0.02 | | | |
| Tocopherol | | | 0.1 | | |
| Retinyl palmitate | | | | 0.2 | |
| Tocopheryl acetate | | 1 | | | |
| Retinol | | | | 0.2 | |
| Ascorbic acid | | | 0.05 | | |
| Salicylic acid | 0.25 | | | 2 | 0.25 |
| Glycolic acid | | 2 | 4 | | |
| Lactic acid | | | 1 | 1 | |
| Glycyrrhiza inflata | 0.025 | | | | 0.025 |
| Bisabolol | | 0.5 | | | |
| Diclofenac | | | 0.1 | | |
| Ibuprofen | | | | 0.2 | |
| Octadecenedioic acid | 0.1 | | 0.5 | | 1 |
| Tretinoin | | | | 0.05 | |
| Azelaic acid | | 10 | | | |
| Glycine Soya | | | 2 | | |
| Trisodium EDTA | 1 | 0.2 | | | 1 |
| Phenoxyethanol | 0.4 | 0.4 | 0.5 | | 0.5 |
| Ethyl paraben | 0.05 | 0.1 | | 0.4 | 0.2 |
| Methyl paraben | 0.25 | | 0.2 | | 0.3 |
| Propyl paraben | 0.05 | 0.1 | | | 0.15 |
| Hexamidine diisethionate | 0.5 | | 0.1 | | |
| DMDM hydantoin | | 0.2 | | 0.1 | |
| Iodopropynylbutyl carbamate | | | 0.25 | | |
| 2-Ethylhexylglycerin ether | | | | 0.4 | |
| (Octoxyglycerin) | | | | | |
| Xanthan gum | 0.1 | 0.1 | | | 0.4 |
| Polyacrylic acid (Carbomer) | | | 0.1 | | |
| Aluminum starch octenylsuccinate | 2 | | 4 | | |
| Chondrus Crispus | 0.25 | 0.2 | | | 0.25 |
| Glycerin | 5 | 5 | 6 | 4 | 1 |
| Butylene glycol dicaprylate/dicaprate | | | | 2 | 3 |
| Water-soluble and/or oil-soluble dyes | | 0.5 | | | |
| Additives (distarch phosphate, SiO₂, talc, | | 0.1 | 0.05 | 3 | |
| BHT) | | | | | |
| Sodium hydroxide | q.s. | q.s. | q.s. | q.s. | q.s. |
| Perfume | q.s. | q.s. | q.s. | q.s. | q.s. |
| Water | ad | ad | ad | ad 100 | ad 100 |
| | 100 | 100 | 100 | | |

### Example 11

### W/O Cream

| | |
|---|---|
| Polyglyceryl-3-diisostearate | 5.0 |
| Polyglyceryl-2-dipolyhydroxystearate | 2.5 |
| Cetearyl alcohol | 2 |
| Cetyl alcohol | 2 |
| C_{12–15} Alkyl benzoate | 8 |
| Caprylic/capric triglyceride | 6 |
| Octyldodecanol | 5 |
| Octamethyltetrasiloxane (Cyclomethicone) | 2 |
| Salicylic acid | 1 |
| TiO₂ | 2 |
| Ethylhexyl methoxycinnamate | 5 |
| Glycyrrhiza inflata root extract | 0.03 |
| Octadecenedioic acid | 0.1 |
| Citric acid, sodium salt | 0.5 |
| Ubiquinone (Q10) | 0.05 |
| Phenoxyethanol | 0.1 |
| p-Hydroxybenzoic acid alkylester (Paraben) | 0.1 |
| Glycerin | 7.5 |
| Fillers (distarch phosphate, SiO₂, talc, aluminum stearate) | 0.2 |
| Perfume | q.s. |
| Water | ad 100 |

### Formula Examples Foundations

| | Example No. | | | |
|---|---|---|---|---|
| | 1 | 2 | 3 | 4 |
| PEG-30-stearate | 1.5 | | | |
| Glyceryl stearate | 0.5 | | | 2 |
| Ceteareth-20 | 1 | | | |
| Polyglyceryl-3 | | | 3.5 | |
| methylglucose stearate | | | | |
| Sorbic acid stearate | | | 1.5 | |
| Stearic acid | 2 | | | 1.8 |
| Glyceryl stearate citrate | | 3.5 | | |
| Cetyl alcohol | 0.5 | 0.75 | 1.0 | |
| Lecithin | | | | 0.5 |
| Veegum K = Mg—Al-silicate | 0.8 | | | 1 |
| Xanthan gum | 0.2 | | | 0.3 |
| Carbomer | | 0.2 | | |
| Hydroxyethyl cellulose | 0.2 | | 0.1 | |
| Caprylic/capric triglyceride | | 2 | 3 | 2 |
| Dicaprylylether | | 2 | 3 | 3 |
| Octyldodecanol | | | 3 | |
| Dimethicone | 3 | | 3 | 3 |
| Cyclomethicone | | 4 | 3 | 2 |
| C_{12–15} Alkyl benzoate | 2 | | | |
| Cetearyl octanoate | 2 | | | |
| Squalane | 1 | | | 6 |
| Isopropyl palmitate | 1 | | | 2 |
| PPG-15 Stearyl ether | 2 | 3 | | |
| Hydrogenated coconut glycerides | 2 | | | |
| Stearyldimethicone | 9 | | | |
| Acetylated lanolin oil | 2 | 0.2 | | |
| Ethylhexyl methoxycinnamate | | | 2 | |
| Ethylhexyl cyanodiphenylacrylate | | | 6 | |
| (Octocrylene) | | | | |
| Bis-ethylhexyloxyphenol | | | 2 | |
| methoxyphenyltriazine | | | | |
| Salicylic acid | 0.2 | 0.5 | | 1 |
| Glycolic acid | | | 2 | |
| Ubiquinone (Q10) | | 0.03 | 0.02 | |
| Retinol | 0.1 | | | |
| Tocopherol | | 0.025 | | 0.5 |
| Tocopheryl acetate | | | 1 | 0.5 |
| Silicone | | 0.8 | | |
| Nylon-12 | | | 5 | |
| Lauroyl lysine | 0.5 | | 0.5 | |
| Kaolin | 0.5 | | 1 | 2 |
| Sodium starch octenylsuccinate | | 1 | | 1.5 |
| Iron oxides | 1.2 | 2.4 | 2.6 | 3 |
| Titanium dioxide (>100 nm) | 3.8 | 5.6 | 4.5 | 4.5 |
| Titanium dioxide (<100 nm) | 2 | | | 2 |
| Interference pigments | 0.2 | | | 0.5 |
| Pigment low luster | 0.2 | | | 0.2 |
| ZnO | | 1 | 2 | |
| Ethylhexyl salicylate | 4 | | 2 | |
| Ethylhexyl methoxycinnamate | | 5 | 4 | 2 |
| Polymethylsilsesquioxane | | 2 | | |
| (Tospearl 2000B) | | | | |
| Octadecenedioic acid | 0.1 | 0.1 | | 0.1 |
| Kojic acid | | | 1 | |
| Glycyrrhiza inflata root extract | | 0.025 | 0.05 | |
| Arctium lappa | 1 | | | 0.5 |
| EDTA | 0.1 | 0.6 | 1 | 1 |
| Glycerin | 2 | 5 | 5 | 10 |
| Phenoxyethanol and | 1 | 0.5 | | 1 |
| Paraben (Phenonip) | | | | |
| Imidazolidinyl urea | 0.3 | 0.3 | | 0.25 |
| Neutralizers | q.s. | q.s. | q.s. | q.s. |
| Perfume, antioxidants | q.s. | q.s. | q.s. | q.s. |
| Water | ad 100 | ad 100 | ad 100 | ad 100 |

It is noted that the foregoing examples have been provided merely for the purpose of explanation and are in no way to be construed as limiting of the present invention. While the present invention has been described with reference to an exemplary embodiment, it is understood that the words which have been used herein are words of description and illustration, rather than words of limitation. Changes may be made, within the purview of the appended claims, as presently stated and as amended, without departing from the scope and spirit of the present invention in its aspects. Although the present invention has been described herein with reference to particular means, materials and embodiments, the present invention is not intended to be limited to the particulars disclosed herein; rather, the present invention extends to all functionally equivalent structures, methods and uses, such as are within the scope of the appended claims.

What is claimed is:

1. A cosmetic preparation comprising
(a) one or more compounds selected from salicylic acid and salts thereof and glycolic acid and salts thereof;
(b) one or more pigments selected from titanium dioxide pigments and zinc oxide pigments.

2. The preparation of claim 1, wherein the preparation comprises (a) in a total concentration of from about 0.05% to about 10% by weight, based on a total weight of the preparation.

3. The preparation of claim 2, wherein the preparation comprises (a) in a total concentration of from about 0.1% to about 4% by weight.

4. The preparation of claim 2, wherein the preparation comprises at least one of salicylic acid and a salt thereof.

5. The preparation of claim 1, wherein the preparation comprises (b) in a total concentration of from about 0.05% to about 10% by weight, based on a total weight of the preparation.

6. The preparation of claim 5, wherein the preparation comprises one or more titanium dioxide pigments in a total concentration of from about 0.1% to about 10% by weight.

7. The preparation of claim 6, wherein the preparation comprises the one or more titanium dioxide pigments in a total concentration of from about 0.5% to about 6% by weight.

8. The preparation of claim 1, wherein (b) comprises one or more titanium dioxide pigments having at least one of a rutile structure, an average particle size of from about 10 to about 100 nm and a specific surface area of from about 30 to about 110 m²/g.

9. The preparation of claim 8, wherein (b) comprises one or more titanium dioxide pigments which have a rutile structure and a specific surface area of from about 70 to about 120 m²/g and are coated with at least one of alumina and simethicone.

10. The preparation of claim 1, wherein the preparation further comprises one or more UV filters selected from phenylene-1,4-bis-(2-benzimidazyl)-3,3′-5,5′-tetrasulfonic acid salts; 2-phenylbenzimidazole-5-sulfonic acid salts; 1,4-di(2-oxo-10-sulfo-3-bomylidenemethyl)-benzene and salts thereof; 4-(2-oxo-3-bomylidenemethyl)benzenesulfonic acid salts; 2-methyl-5-(2-oxo-3-bomylidenemethyl)sulfonic acid salts; 2,2′-methylene-bis-(6-(2H-benzotriazole-2-yl)-4-(1,1,3,3-tetramethylbutyl)-phenol); 2-(2H-benzotriazole-2-yl)-4-methyl-6-[2-methyl-3-[1,3,3,3-tetramethyl-1-[(trimethylsilyl)-oxy]disiloxanyl]propyl]-phenol; 3-(4-methylbenzylidene)camphor; 3-benzylidenecampher; ethylhexyl salicylate; terephthalidene dicamphorsulfonic acid; (2-ethylhexyl)4-(dimethylamino)benzoate; amyl 4-(dimethylamino)benzoate; di(2-ethylhexyl)4-methoxybenzalmalonate; (2-ethylhexyl)4-methoxycinnamate; amyl 4-methoxycinnamate; 2-hydroxy-4-methoxybenzophenone, 2-hydroxy-4-methoxy-4′-methylbenzophenone; 2,2′-dihydroxy-4-methoxybenzophenone; hexyl 2-(4′-diethylamino-2′-hydoxybenzoyl)benzoate, 4-(tert-butyl)-4′-methoxydibenzoylmethane; homomenthylsalicylate; 2-ethylhexyl 2-hydroxybenzoate; 2-ethylhexyl 2-cyano-3,3-diphenylacrylate; dimethicodiethylbenzalmalonate; 3-(4-(2,2-bisethoxycarbonylvinyl)-phenoxy)propenyl)-methoxysiloxane/dimethylsiloxane co-polymer; 2,4-bis-{[4-(2-ethyl-hexyloxy)-2-hydroxy]-phenyl}-6-(4-methoxyphenyl)-1,3,5-triazine (INCI: Bis-Ethylhexyloxyphenol Methoxyphenyl Triazine); dioctylbutylamidotriazone (INCI: Diethylhexyl Butamidotriazone); 2,4-bis-[5-1(di-methylpropyl)benzoxazole-2-yl-(4-phenyl)-imino]-6-(2-ethylhexyl)-imino-1,3,5-triazine (CAS No. 288254-16-0); tris(2-ethylhexyl)4,4′,4″-(1,3,5-triazine-2,4,6-triyltriimino)-tris-benzoate (also: 2,4,6-Tris-[anilino-(p-carbo-2′-ethyl-1′-hexyloxy)]-1,3,5-triazine (INCI: Ethylhexyl Triazone); 2,4,6-tribiphenyl-4-yl-1,3,5-triazine; merocyanine; benzoylcinnamic acid nitrites in a concentration of from about 0.01% to about 40% by weight, based on a total weight of the preparation.

11. The preparation of claim 1, wherein the preparation further comprises one or more antioxidants in a total concentration of from about 0.01% to about 10% by weight, based on a total weight of the preparation.

12. The preparation of claim 11, wherein the one or more antioxidants are selected from hydroquinone, hydroquinone derivatives, tocopherol, tocopheryl derivatives, ascorbic acid and ascorbyl derivatives.

13. The preparation of claim 12, wherein the one or more antioxidants are present in a total concentration of from about 0.01% to about 5% by weight.

14. The preparation of claim 1, wherein the preparation further comprises one or more melanogenesis reducing substances in a total concentration of from about 0.01% to about 10% by weight, based on a total weight of the preparation.

15. The preparation of claim 14, wherein the one or more melanogenesis reducing substances comprise at least one substance selected from octadecenedioic acid, kojic acid, soy, α-lipoic acid, retinoids, azelaic acid and phenolic compounds.

16. The preparation of claim 15, wherein the one or more melanogenesis reducing substances comprise octadecenedioic acid and are present in a total concentration of from about 0.1% to about 2% by weight.

17. The preparation of claim 1, wherein the preparation further comprises one or more anti-inflammatory substances in a concentration of from about 0.01% to about 5% by weight, based on a total weight of the preparation.

18. The preparation of claim 17, wherein the one or more anti-inflammatory substances comprise at least one extract of glycyrrhiza inflata, chamomilla recutita or arctiium lappa.

19. The preparation of claim 1, wherein the preparation comprises (i) one or more titanium dioxide pigments in a concentration of from about 0.1% to about 10% by weight;
(ii) at least one of salicylic acid and a salt thereof in a concentration of from about 0.05% to about 10% by weight;
(iii) one or more antioxidants selected from ubiquinone, tocopherol and tocopherol derivatives in a concentration of from about 0.005% to about 7% by weight;
(iv) octadecenedioic acid in a concentration of from about 0.05% to about 5% by weight;
each based on a total weight of the preparation.

20. The preparation of claim 1, wherein the preparation comprises
(i) one or more titanium dioxide pigments having a rutile structure in a concentration of from about 0.1% to about 10% by weight;
(ii) at least one of salicylic acid and a salt thereof in a concentration of from about 0.1% to about 2% by weight;
(iii) ubiquinone in a concentration of from about 0.01% to about 0.5% by weight;
(iv) octadecenedioic acid in a concentration of from about 0.1% to about 2% by weight;
(v) glycyrrhiza inflata in a concentration of from about 0.01% to about 1% by weight;
each based on a total weight of the preparation.

21. The preparation of claim 20, wherein the preparation further comprises at least one of ethylhexyl methoxycinnamate, ethylhexyl salicylate, benzophenone-3, phenylbenzimidazole sulfonic acid and homosalate.

22. A cosmetic preparation comprising
(a) from about 0.1% to about 4% by weight of one or more compounds selected from salicylic acid and salts thereof;
(b) from about 0.5% to about 6% by weight of one or more titanium dioxide pigments;
each based on a total weight of the preparation.

23. The preparation of claim 22, wherein (b) comprises one or more titanium dioxide pigments having a rutile structure and an average particle size of from about 10 to about 100 nm.

24. The preparation of claim 23, wherein (b) comprises one or more titanium dioxide pigments having a specific surface area of from about 30 to about 110 m²/g.

25. The preparation of claim 22, wherein (b) comprises one or more titanium dioxide pigments which have a rutile structure and a specific surface area of from about 70 to about 120 m²/g and are coated with alumina and simethicone.

26. The preparation of claim 22, wherein the preparation further comprises one or more antioxidants selected from ubiquinone, tocopherol and tocopherol derivatives in a total concentration of from about 0.01% to about 10% by weight, based on a total weight of the preparation.

27. The preparation of claim 22, wherein the preparation further comprises one or more melanogenesis reducing substances selected from octadecenedioic acid, kojic acid, soy, α-lipoic acid, retinoids, azelaic acid and phenolic compounds in a total concentration of from about 0.01% to about 10% by weight, based on a total weight of the preparation.

28. A cosmetic preparation comprising
(a) from about 0.1% to about 4% by weight of one or more compounds selected from salicylic acid and salts thereof;
(b) from about 0.5% to about 6% by weight of one or more titanium dioxide pigments having a rutile structure and a specific surface area of from about 70 to about 130 m²/g;
and at least one of
(c) from about 0.01% to about 5% by weight of one or more antioxidants selected from ubiquinone, tocopherol and tocopherol derivatives;
(d) from about 0.1% to about 2% by weight of one or more substances selected from octadecenedioic acid, kojic acid, soy, α-lipoic acid, retinoids, azelaic acid and phenolic compounds; and
(e) from about 0.01% to about 3% by weight of one or more anti-inflammatory substances;
each based on a total weight of the preparation.

29. A method of preventing or treating skin pigmentation, wherein the method comprises applying to skin the cosmetic preparation of claim 1.
